(19) United States
(12) Patent Application Publication
(10) Pub. No.: US 2009/0227927 A1
(43) Pub. Date: Sep. 10, 2009

(54) Orthopedic walking brace

(76) Inventor: Michael J. Frazer, Minneapolis, MN (US)

Correspondence Address: MERCHANT & GOULD PC, P.O. BOX 2903, MINNEAPOLIS, MN, 55402-0903, US

(21) Appl. No.: 12/381,371

(22) Filed: Mar. 10, 2009

Related U.S. Application Data
(60) Provisional application No. 61/068,959, filed on Mar. 10, 2008.

(51) Int. Cl. A61F 5/00

(52) U.S. Cl. 602/27

(57) ABSTRACT

An improved orthopedic walking brace for providing therapeutic pressure to an ankle and lower leg is provided. An exterior stirrup frame and detachable front tongue and rear panel members apply compressive forces through an inner liner member to the foot, ankle and leg. Inflatable bladders are positionable adjacent the ankle for providing adjustable pressure. The air inlet tubes for the bladders are adjustable to an infinite number of positions relative to the stirrup member.

### CROSS REFERENCE TO RELATED APPLICATIONS

This application acclaims priority to U.S. Provisional Patent Application Ser. No. 61/068,959 filed on Mar. 10, 2008, the entire contents of which are herein incorporated by reference.

### FIELD OF THE INVENTION

This invention relates generally to orthopedic devices, and more particularly to orthopedic walker support boots which are configured to immobilize and/or protect injured limbs or parts of the anatomy for use by persons recovering from injuries such as broken bones, sprains or other trauma of the lower leg, ankle or foot.

### BACKGROUND OF THE INVENTION

Orthotic or orthopedic devices to which this invention relates function to brace and support injured limbs, muscles, joints and bones. Such injuries can arise in numerous ways, such as through strenuous physical activity or from weakened conditions that may be experienced by frail or elderly people. In the management of certain such injuries to the lower extremities, such as fractures to the tibia and fibula, malleolar fractures, or severe ankle sprains, it is common to first completely immobilize the lower extremity by use of well-known molded plastic or resin casts. However, once the injured extremity has become stable, it has been found that recovery can be advanced more rapidly by gradually and progressively permitting the extremity to bear weight and undergo other permitted exercises. It has been found that non-cast devices typically known as walking boots or braces are particularly effective for filling this need. Besides aiding in the healing process, they provide the wearer with significantly increased mobility, greater comfort and the ability to bathe and readily examine the injured extremity.

Many configurations of such walking braces are known in the art. One such early device disclosed, for example, in U.S. Pat. No. 3,955,565 to Johnson, comprises a pair of complimentary shaped half shell members that are configured to be secured to one another in enclosing relation about a damaged limb, to form an enclosed boot-like structure. Another example of a walker boot of such cooperating front/back two part construction, is shown in U.S. Pat. No. 5,827,210 to Antar et al.

Other such devices such as, for example, that of U.S. Pat. Nos. 5,078,128 or 5,464,385 to Grim, employ a lighter weight frame construction generally including a load bearing sole structure for supporting the foot, connected to a pair of lateral support members extending upwardly from the sole along the inner and outer sides of the ankle or leg to form a stirrup-like structure. The wearer's foot and injured limb are supported in a padded liner member that is retained within the outer stirrup frame structure by means of a plurality of strap fasteners. The referenced walker devices are generally open at the front and back of the stirrup frame, except for the support liner and the fastening straps. One or more inflatable bladders have been used with the liner or frame structure to selectively apply pressure to the retained limb(s) of the wearer.

U.S. Pat. No. 5,577,998 to Johnson et al. illustrates a walking brace structure having a rear shell portion that appears to be of the open back stirrup configuration, an interior resilient foam liner, a front shell portion and a plurality of straps for securing the assembly about the wearer's leg. Johnson further discloses an air cell structure that can be inflated without the use of an external pump.

The present invention provides a walking brace that offers advantages over those of the prior art. The walker of this invention provides the support benefits of known walker devices with a lightweight yet strong and fully protective support structure that offers adjustable air bladder comfort features to the wearer heretofore not found in prior walkers.

### SUMMARY OF THE INVENTION

The invention provides an improved therapeutic walking brace in the form of a walking boot with a rocker sole that effectively immobilized the foot and ankle of a patient/wearer while allowing the wearer to walk without pain. An internal padded line member wraps around the wearer's foot and ankle. Immobilizing pressure is applied to the foot, ankle and leg of the wearer through the liner by an external framework of a sole and stirrup configuration and front tongue and back panel members. The external force applying members are adjustably secured together by a plurality of adjustable straps. A pair of fluid bladders are positioned within pocket members of the liner at positions alongside the inner and outer ankle portions of the wearer. The bladders are readily removable from their support pockets and include inflation tubes that extend upwardly to the top of the liner for easy inflation of the bladders. The inflation tubes are configured to be positioned at an infinite number of positions relative to the ankle and external stirrup framework to provide for maximum comfort to the wearer.

According to one aspect of the invention there is provided, a walking brace for providing therapeutic pressure to the ankle and lower leg of a person, comprising:
a. an exterior stirrup frame construction, comprising:
   i. a full length sole portion; and
   ii. a pair of rigid or semirigid generally parallel elongate strut members extending substantially vertically upward from said sole portion on either side thereof so as to extend along an ankle and leg of a person wearing said brace;
b. an interior resilient liner pad configured to cushion the ankle and leg of a person and to cooperatively position the leg within said stirrup frame;
c. at least one detachable variable pressure fluid bladder;
d. at least one bladder support pocket attached to said liner pad configured to support said at least one fluid bladder, said support pocket having an access suitable for enabling ingress and egress of said bladder therethrough;
e. a fluid inlet tube operatively connected to and extendable from said fluid bladder to a position remote therefrom and accessible externally of said liner and said stirrup frame; and
f. a fastener operatively connectable to said inlet tube to selectively secure said inlet tube at a plurality of locations relative to said liner and said stirrup frame.

According to another aspect of the invention there is provided, an inflatable fluid bladder support member for selectively applying pressure to an ankle and leg of a patient and of a type suitable for placement between an outer shell of an orthopedic brace member and the ankle or leg of a patient, comprising:
a. a fluid inflatable bladder; having an internal cavity;
b. an inlet tube having a first end operatively connected to said bladder in fluid communication with said bladder internal cavity, and extending to an opposite second end suitable for selective attachment to a pump; and
c. a fastener member secured to said tube between said first and second ends thereof and having an outer surface suitable for detachable connection to a second attachment surface; said outer surface being attachable at an infinite number of positions along said second attachment surface.

These and other aspects of the invention will become more apparent upon a description of a preferred embodiment of the invention. It will be appreciated that the preferred embodiment is not to be construed as limiting the invention to any particular configuration, designs, or applications that are specifically presented herein. The preferred embodiment is presented to illustrate a specific application and implantation of the broader principles of the invention and is not to be construed in a limiting manner.

### BRIEF DESCRIPTION OF THE DRAWINGS

Referring to the Figures of the Drawing, wherein like numerals represent like parts throughout the several views:

FIG. 1 is a top, front, perspective view of a preferred embodiment of a right foot walking brace of the present invention, illustrated as it would appear assembled and positioned around the foot and leg of a wearer;

FIG. 2 is a top, front, perspective view of the outer stirrup portion of the brace of FIG. 1;

FIG. 3 is a perspective view illustrating the front tongue and pad, and back panel member portion of the brace of FIG. 1;

FIG. 4 is a left side view of the inner liner portion of the brace of FIG. 1, shown removed from the outer stirrup member, and illustrating an outer pocket and an air bladder assembly operatively positioned therein.

FIG. 5 is a diagrammatic perspective view of the inner liner of FIG. 4, illustrating the liner as it would appear in an unfolded, open position relative to the outer stirrup prior to insertion of a foot/leg therein;

FIG. 6 is an enlarged exploded view of the inside portion of the right upright stirrup support member of FIGS. 1 and 2, illustrating the interconnection of the upper and lower portions thereof;

FIG. 7 is a diagrammatic view of a typical placement of the air bladder of FIG. 4 relative to a foot and ankle of a wearer of the brace of FIG. 1; and

FIG. 8 is an enlarged fragmented view of the air bladder assembly of FIGS. 4 and 7, illustrating its connection with a pump.

### DETAILED DESCRIPTION OF THE PREFERRED EMBODIMENT

Referring to the drawings, wherein like numerals represent like parts throughout the several views, FIGS. 1-8 illustrate a first embodiment of a walker brace 10 constructed according to the principles of this invention. The walker brace is shown assembled in FIG. 1 as it would appear when secured to the foot and leg of a patient (not illustrated). The outer shell portions of the walker brace are preferably constructed of a light weight plastic material suitable for use with injection molding equipment. The outer shell portions of the walker brace include a stirrup-like frame portion 12, front tongue member 35 and a rear panel member 40.

The stirrup-like frame portion 12 includes a lower foot support full length sole portion 13 for supporting the bottom of the wearer's foot. The back portion of the lower foot support 13 defines a slightly raised heel portion 14, continuously connected with opposed inner and outer side portions 15 and 16 respectively which rise to form a pair of inner and outer strut base members 15a and 16a respectively configured to lie along the lower ankle portions of a wearer. The walker brace illustrated in the figures is a “right” foot brace. A left foot brace would comprise a mirror image of the walker brace illustrated in the figures. Accordingly, the previously described inner side 15 of the lower foot support portion would correspond to the left side of the right foot walker brace member when positioned on a user's foot; and the outer side 16 would correspond to the right side of the illustrated right foot walker brace when positioned on a user's foot. The inner/left side and the outer/right side terminology will be used interchangeably throughout the description, it being understood that such reference is to a right foot walker brace member of the preferred embodiment illustrated. It will also be understood that the same principles as described with respect to the right foot walker brace, apply to a walker brace design for a wearer's left foot. The lower foot support sole portion defines a fairly wide footbed to provide comfortable fit to the wearer. A footbed foam cushion 17 provides improved energy absorption and reduces shock input to the heel and foot of the wearer. The raised heel portion 14 is configured to engage the back of the heel of the wearer and to define the rearward extent of a foot position when secured within the walker brace. The outer bottom 13 of the foot support sole portion is continuously curved to provide a “rocker” bottom to facilitate a more natural walking gait pattern for the wearer. The lower surface of the sole portion has a patterned rubberized tread (not illustrated) as is known in the art to provide an anti-slip walking surface for the brace. Other materials such as gel or foam, or air bladder structures may be used for forming the footbed cushion 17 of the walker brace, as will be appreciated by those skilled in the art.

A left side elongate strut extension 15b is secured to the lower upright strut brace member 15a. Similarly, a right side elongate strut extension 16b is secured to the lower upright strut brace member 16a. The strut brace member 15a and its elongate strut extension 15b form a left side upright support member for the walker brace. Similarly, the upright strut brace member 16a and its associated elongate extension 16b form a right side upright support member of the walking brace. The left and right upright support members, in combination with the lower foot support sole portion 13, the heel portion 14, and the left and right outer side portions 15 and 16 collectively form a stirrup-like outer support structure for the walker brace 10.

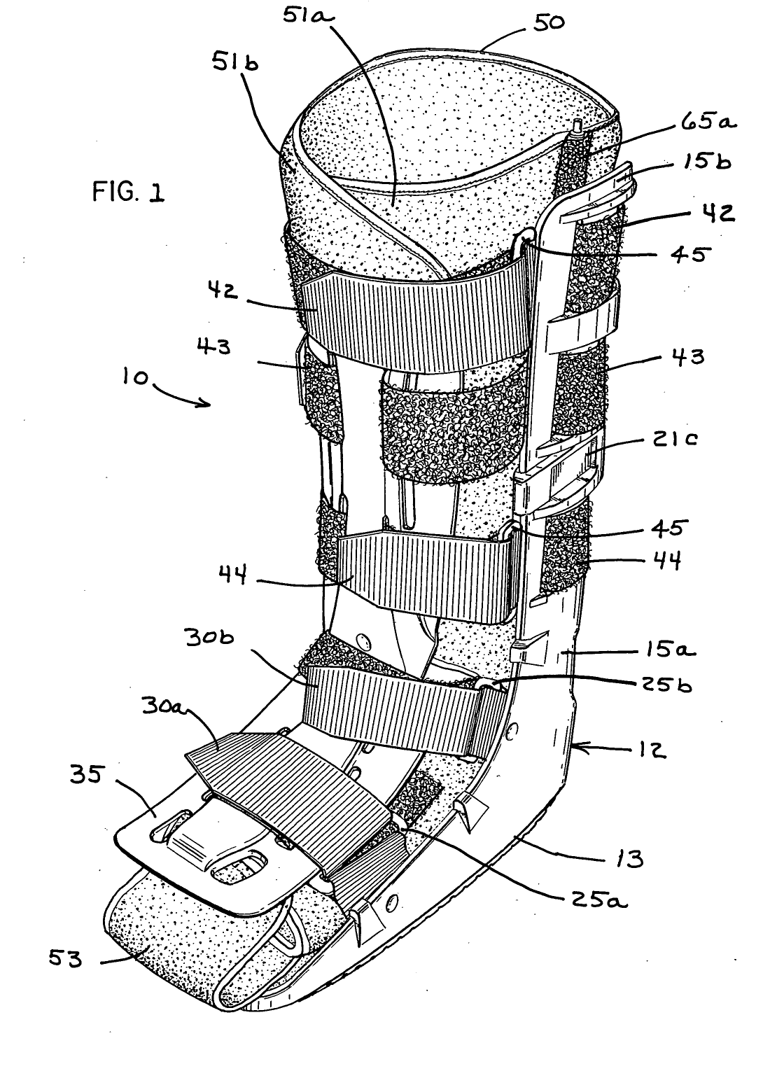

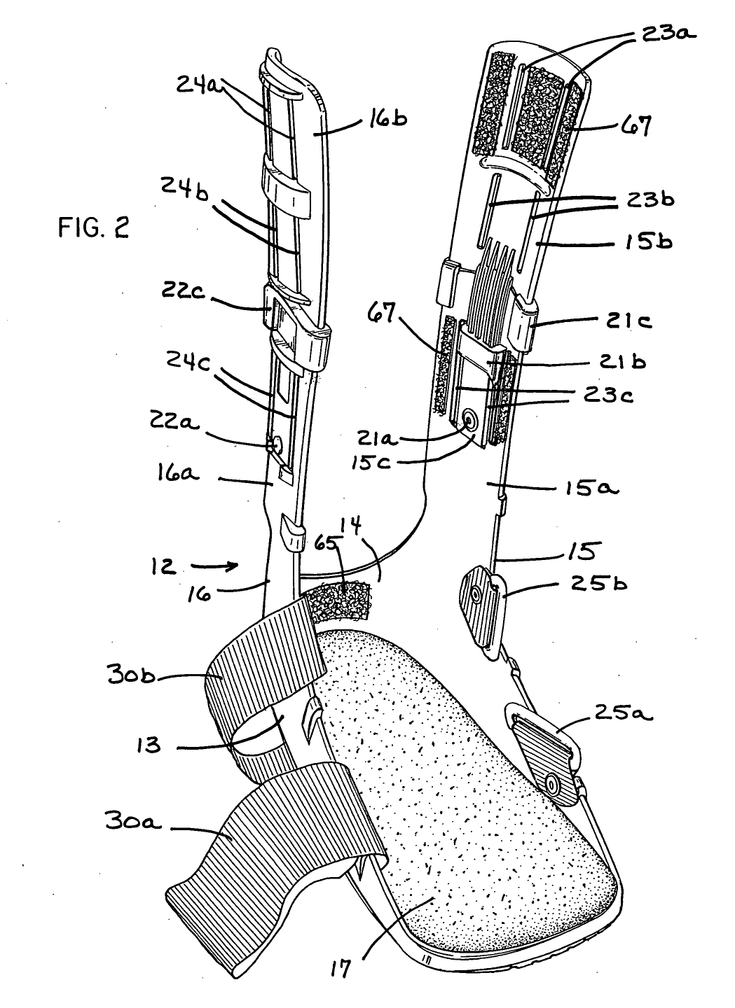

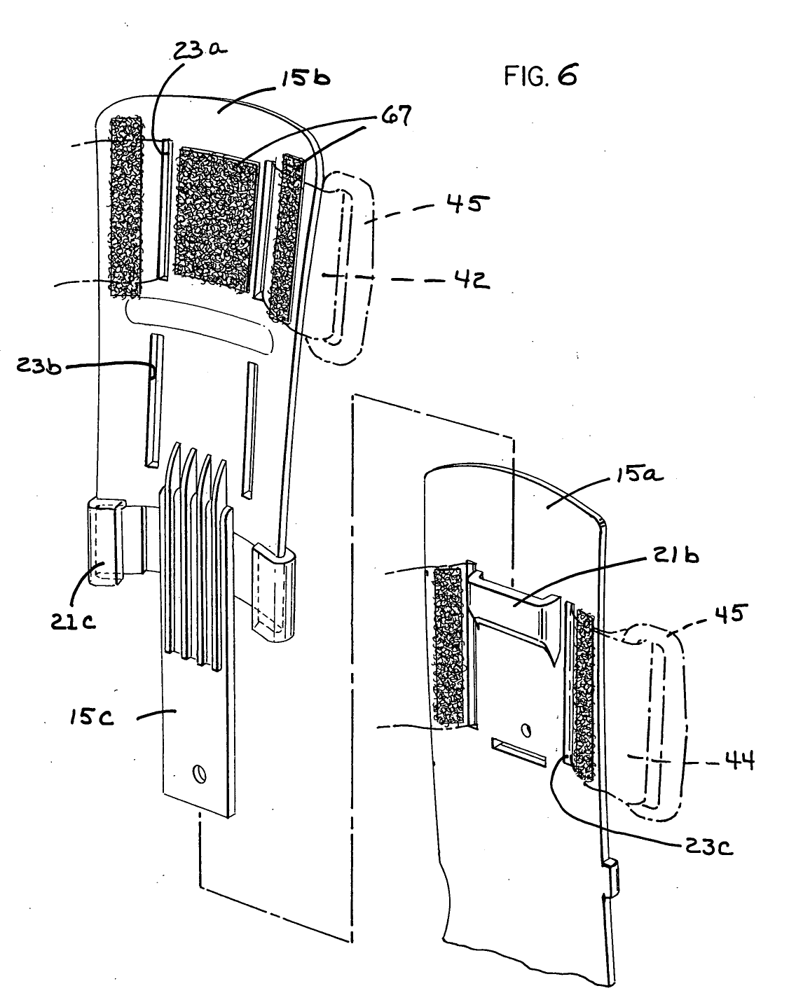

The left side strut extension 15b overlaps with the upright strut brace member 15a and is secured thereto by a rivet 21a, an inner mounting band 21b inwardly projecting from the left side upright strut brace member 15a and an outer collar sleeve member 21c that slides over the upper edge of the lower strut brace member 15a (see FIGS. 2 and 6). Similarly, the right side elongate strut extension 16b is secured to the upright strut brace member 16a by means of a rivet 22a, an inner mounting band 22b (not shown) inwardly projecting from the right upright strut brace member 16a and an outer collar sleeve member 22c that slides over the upper edge of the lower strut brace member 16a. Each of the upper strut extension tongue portion 15c and 16 respectively the cooperatively slides through and is retained by the inner mounting bands 21b and 22c of the lower strut brace member 15a and 16a respectively. The pair of elongate upright members 15a/b and 16a/b form semirigid support for the inner and outer sides of the wearer's ankle/leg members for the brace. The semirigid support members are longitudinally anatomically contoured to accommodate the general shape of a wearer's ankle and leg that they will support.

The left side elongate upright support member 15a,b of the stirrup includes three pairs of longitudinally spaced slots 23a, 23b and 23c through which are retainably threaded fastener straps, as described in more detail hereinafter. The right side elongate upright support member 16a,b of the stirrup includes three pairs of longitudinally spaced slots 24a, 24b and 24c through which are retainably threaded fastener straps, as described in more detail hereinafter.

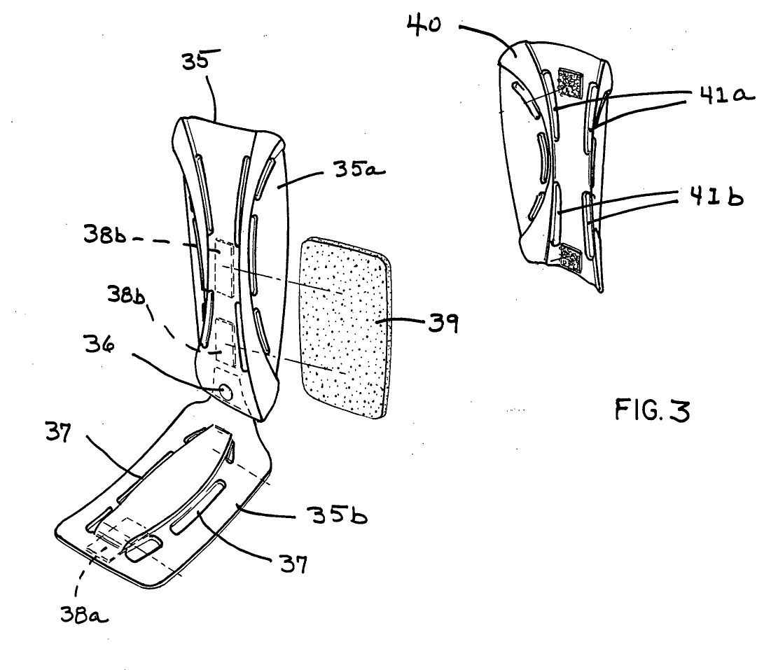

Referring to FIG. 2, first and second D fastening rings 25a and 25b are pivotally mounted by rivet fasteners to the left or inner side portion 15 of the lower foot support portion 13 of the brace. A pair of fastening straps 30a and 30b are pivotally secured by means of rivets to the outer side 16 of the lower foot support sole portion 13 at positions therealong which cooperatively laterally align with the D-rings 25a and 25b respectively such that the strap members 30a and 30b can be threaded through the D-ring fasteners 25a and 25b respectively and folded back upon themselves to form securing fastener strap members in a manner well-known in the art. The outer surfaces of the strap members 30a and 30b include appropriately positioned hook and loop fastener material to enable fastening of the strap members 30a and 30b when folded back upon themselves. The inside surfaces of the outer shell portions include hook-type Velcro fastener tabs positioned for securing to a removable inner liner, as hereinafter described in more detail. Referring to FIG. 3, a front tongue member 35 is in the preferred embodiment constructed of an upper portion 35a and a lower portion 35b, pivotally connected by means of a rivet 36. The lower portion 35b includes a pair of laterally spaced slots 37 which could be sized to threadingly accommodate the first fastening strap 30a therethrough. The back side of the tongue 35 includes a plurality of hook-type fastener members 38. The lowermost fastener member 38a on the lower tongue portion 35b is configured to hold the lower tongue portion 35b in position relative to the toe portion of the inner liner member. The upper fastener members 38b secure a pad member 39 to the inner surface of the upper tongue portion 35a.

The rear panel member 40 (FIG. 3) includes two pairs of laterally spaced slots 41a and 41b. The slots 41a are longitudinally spaced from the slots 41b along the rear panel member 40 and positioned therealong to cooperatively accept fastener straps therethrough. When operatively connected by the fastener straps, the slots 41a generally vertically align with the slots 23b and 24b of the upright stirrup support members, and the slots 41b generally vertically align with the slots 23c and 24c of the upright supports of the stirrup assembly.

Upper fastener straps 42, 43, and 44 secure the left and right upright stirrup members, the front tongue 35 and the rear panel member 40 to one another and apply pressure through the outer brace framework to the inner liner of the brace. A first end of each of the straps 42, 43, and 44 has a D-ring 45 fastener secured thereto, to enable the opposite free end of the strap to be threaded through the D-ring such that each strap can be secured back upon itself. The upper fastener strap 42 is threaded through the upper slots 23a and 24a in the support struts 15b and 16b respectively. The middle fastener strap 43 is threaded through the slots 24b and 23b of the upper support struts 16b and 15b respectively and also through the upper slots 41a of the rear panel member 40. The lower fastener strap 44 is threaded through the slots 23c and 24c of the upright strut members 15a and 16a respectively, as well as through the lower slots 41b of the rear panel member 40. The straps 43 and 44 may also be optionally threaded through appropriately placed aligned slots within the upper portion 35a of the tongue 35, to fix placement of the upper portion of the tongue 35a relative to the upright support arms of the stirrup portion. As with the lower fastener straps 30a and 30b, the upper fastener straps 42, 43, and 44 include hook and loop fastening materials along their outer surfaces to enable self fastening of the straps back upon themselves in a manner well-known in the art. As shown in the Figures, the straps 42 and 44 are wound around the brace (when viewed from above) in a counter clockwise direction; whereas the middle strap 43 in a clockwise direction.

A removable inner liner wrap member 50 is sized to cooperatively fit within the outer stirrup 12 and is configured to cooperatively encircle the ankle, foot and lower leg portions of the wearer. In the preferred embodiment, the liner 50 is constructed of an ultra plush foam material which offers superior patient comfort. The liner includes thicker padding adjacent the heel area for additional comfort to the heel and protection of the lower portion of the Achilles tendon. The liner member includes a pair of upper flaps 51a and 51b that can be folded forward in overlapping manner around the upper ankle and leg portion of the wearer, and a pair of lower flaps 52a and 52b that can be folded in overlapping manner around the lower foot portion of the wearer. A detachable toe pad 53 is adjustably attachable to the liner member 50 at the forward foot portion of the liner to optionally cover the toes of the wearer. The toe pad 53 is secured to the outer surface of the liner 50 by means of hook-type fastener portions. The liner includes a pair of pockets 56a, 56b positioned along the lower outer sides of the liner 50 for supportively housing a pair of air bladders 58a, 58b respectively. The pockets 56 are configured to position the air bladders 58 adjacent the ankle portions of the wearer for selectively providing therapeutic pressure and/or additional comfort to the ankle portion and to cushion the ankle from the upright support arms of the brace stirrup.

The outer surfaces of the inner liner wrap member 50 comprise a Velcro hook receivable material such as brushed nylon so as to cooperatively receive and fasten to the Velcro hook fasteners of the outer shell portions and tongue and rear panel members so as to cooperatively positionally secure the inner liner member thereto. The inner surface edge of the upper flap portion 51b and the inner edge portion 52b of the lower foot engaging flap each include a small strip of Velcro hook fastener material 51c and 52c enable the upper and lower flaps 51 and 52 respectively to fasten to one another when folded in overlapping manner about the leg and foot portions of the wearer. The pockets 56 for retaining the air bladders are in the preferred embodiment, constructed of four-way expandable elastic material to accommodate expansion of the air bladders 58 inserted therein. Small double-sided adhesive strips (not shown) may be are optionally secured to the outer surfaces of the inner liner 50 near the front and back portions thereof to hold the tongue member 35 and the rear panel member 40 in place against the liner as the brace is being secured to the wearer.

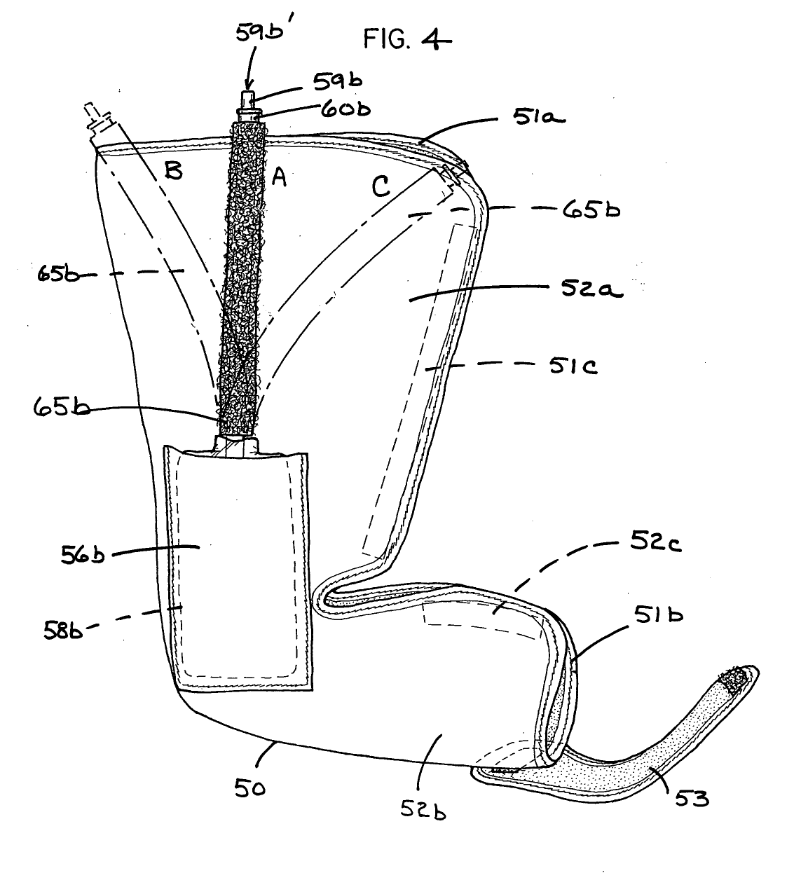

The air bladders 58 may be constructed of any appropriate material suitable for retainably holding a charge of gas or fluid. In the preferred embodiment, the fluid retained by the bladders 58 is air; however, other fluids could be used. Such bladder constructions are well-known in the art and will not be detailed herein. In the preferred embodiment, the retaining pocket 56b on the right outer portion of the liner 50 holds bladder 58b, and the retaining pocket 56a on the left outer portion of the liner 50 holds bladder 58a. Air is supplied to each of the bladders 58a and 58b by means of a plastic air inlet tube 59a and 59b respectively. The outer terminus of the air inlet tubes defines an air inlet port 59a and 59b includes a valve structure of a type well-known in the art, generally indicated at 60a and 60b respectively. The length of the air inlet tubes 59 is sized to extend generally up toward the upper edge of the liner member 50 and configured so as to be accessible by the user when the brace is secured to the wearer's foot and leg, for adjustably pressurizing the air bladder 58. In prior art brace configurations, the position of the air inlet port is generally at a fixed location relative to the walker shell construction. FIG. 4 illustrates the air bladder 58 as being fully inserted within its pocket 56 as it would operatively be positioned when the walker brace is operatively mounted on a patient or wearer. FIG. 8 illustrates the air bladder 58 and it associated air inlet tube 59 as it would appear fully removed from the retaining pocket 56. Removability of the air bladder 58 from their retaining pockets enables ready replacement of the air bladders in the event of failure thereof or other need for replacement of the air bladders, without requiring replacement of the entire liner assembly. FIG. 8 illustrates an air bladder 58 in more detail. FIG. 8 also illustrates a hollow resilient pump bulb 62 of a type well-known in the art, which can be used to inflate the bladder 58 through its air inlet tube 59 and associated valve outlet 60 by a simple squeezing motion, as is well known in the art. The pump bulb 62 is illustrated as it would be operatively connected to the air inlet tube and valve outlet thereof during a pumping operation.

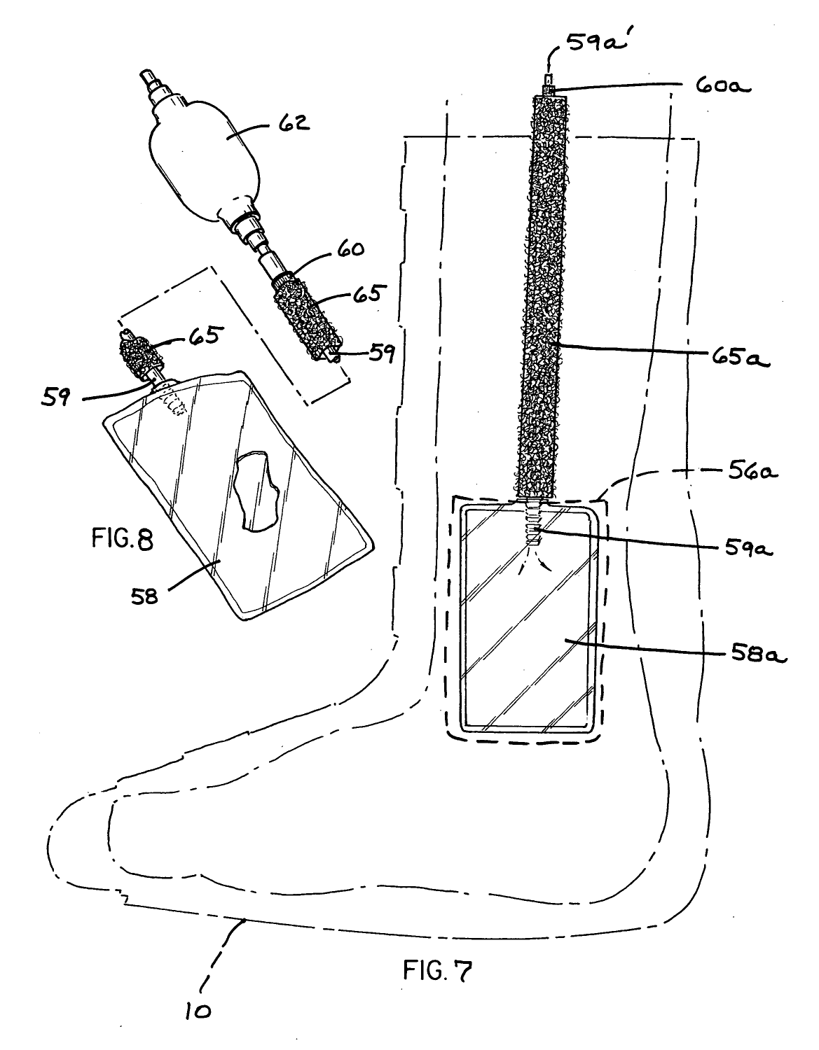

A significant feature of the invention is the ability to adjust the longitudinal and lateral position of the air inlet tubes 59 and their respective valve outlets 60 relative to the side portions of the inner liner 50, and thus relative to the position of the elongate upright support members of the stirrup assembly. The ability to adjust the position of the air inlet tubes 59 enables the wearer to position the valve outlet portions of the inlet tubes at positions that are readily assessable and reachable by the wearer for ease of adjusting the pressure within the bladders 58. Further, the air inlet tubes and respective valve outlets can be positioned so as not to underlie various portions of the upright frame portions of the stirrup shell, for maximizing comfort to the ankle and leg of the wearer. The air inlet tube adjustment feature is in the preferred embodiment provided by means of a pair of Velcro tubular fastener sleeve members 65 in which the air inlet tubes 59 are positioned as illustrated in FIGS. 7 and 8. In the preferred embodiment, that surface of the fastener sleeve 65 which addresses the outer surface of the inner liner 50 is formed of a hook-type Velcro fastener, for cooperatively engaging the outer surface of the liner. The Velcro fastener sleeves 65 enable a wearer to rapidly detach and move the sleeve and attached air inlet tube from the outer surface of the liner 50, to rapidly reposition the fastener sleeve and its attached air inlet tube 59 to the desired position. They also enable the bladder assembly to be rapidly removed and replaced in the liner. FIG. 4 illustrates the air inlet tube adjustability feature provided by the fastener sleeve 65. In FIG. 4, the air inlet tube 59a is illustrated as positioned generally along a center portion of the side of the liner at position “A”. The air inlet tube 59a and its associated valve outlet 60a are shown secured more toward the back portion of the liner 50 at position “B”. The air inlet tube 59a is illustrated as being secured by the fastener sleeve 65a more toward the front portion of the liner assembly 50 at position “C”. It will be appreciated that with an elongated fastener member such as 65, the retained air inlet tube can be continuously positioned in fastened manner relative to the underlying outer surface of the liner 50 in a multitude of arcuate or serpentine configurations. It will be understood that fastener members other than the tubular sleeve configuration depicted at 65 could be used for securing and changing the relative position of the air inlet tubes 59 relative to the liner 50 and thus relative to the stirrup configuration of the walker brace. It will also be appreciated that the length of the air inlet tube is sufficient to allow filling of the bladder with an air pump from external of the brace, when the brace is operatively positioned on a wearer.

It will be appreciated that additional padding materials may be included to add comfort to the wearer. For example, pad 39 (FIG. 3) could be added to the front tongue member 35.

The walker brace may be assembled by first attaching the rear panel member 40 to the upright strut brace members 16a and 16b on the right side of the brace and 15a and 15b on the left side of the brace by means of the fastener straps 43 and 44. Strap 44 is threaded through the slots 23c of the strut 15a, the slots 41b of the rear panel member 40 and slots 24c of the right side strut 16a such that the D ring 45 strap 44 lies adjacent the forward edge of the left strut member 15a. Strap 43 is threaded in the opposite direction, starting with the slots 24b of the right side strut extension 16b, the slots 41a of the rear panel member 40 and the slots 23b of the left side strut extension 15b, such that the D ring 45 thereof lies adjacent the forward edge of the right side strut extension 16b. Straps 43 and 44 thus align and hold the rear panel member 40 in position between the rear edges of the upright stirrup members. The upper strap 42 is threaded through the upper slots 23a of the left strut extension 15b, is positioned over the rearward facing surface of the rear panel member 40, and is threaded through the upper slots 24a of the right side strut extension 16b, such that the D ring 45 thereof lies adjacent the forward edge of the left strut extension 15b.

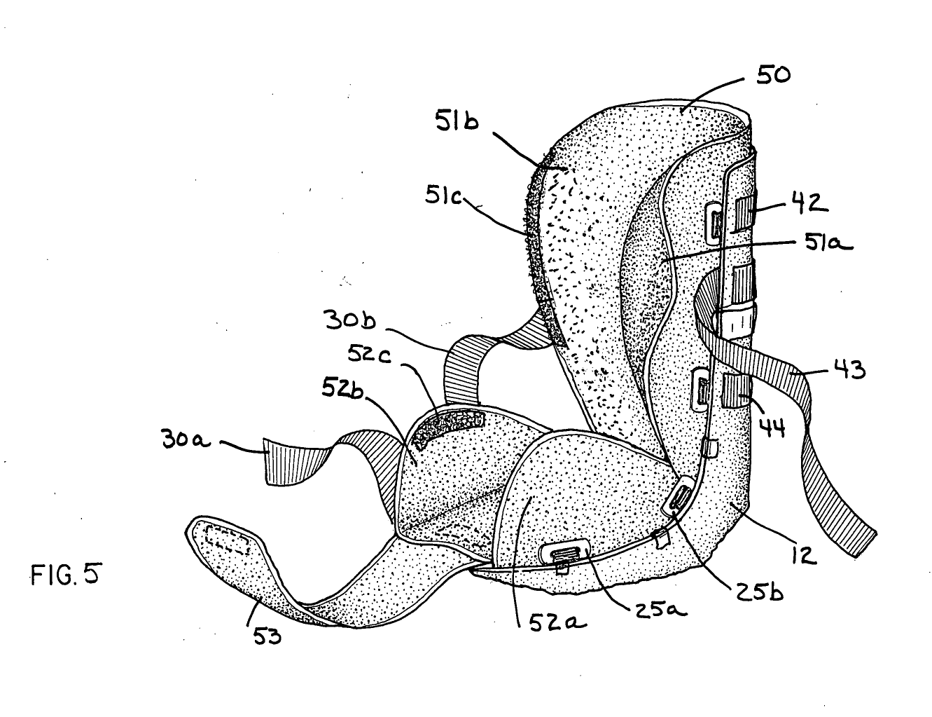

The inner liner 50 is operatively positioned and aligned within the foot support and stirrup-like frame portion 12 in open manner (such as shown in FIG. 5) so as to receive a foot and leg therein. The liner is held in place within the outer framework by a Velcro fastener 65 (FIG. 2) in the inside portion of the heel 14, and by other hook-type fasteners disposed within the inner surfaces of the stirrup assembly as generally shown at 67 in FIG. 2. The toe panel 53 is folded forwardly down to expose and open the front portion of the inner liner 50, and the upper and lower flap panels 51 and 52 of the liner are detached from one another and opened so as to enable access through the top and front of the liner into the inner liner cavity. The wearer's foot is then positioned within the inner liner and moved backward therein such that the wearer's heel lies adjacent the heel portion 14 of the lower foot support 13. If necessary, or desired, additional auxiliary pads (not shown) are positioned as desired relative to the wearer's leg and are secured in the desired position(s), to the inner or outer surfaces of the liner 50.

The upper flaps 51 and the lower flaps 52 are then folded in overlapping manner and respectively secured to one another by their Velcro strips to encircle the wearer's leg and foot portions. The toe portion 53 of the liner is then folded up over the toes of the wearer and fastened to the outer surface of the main liner insert 50. At this point, the Velcro sleeve fasteners 65 can be appropriately adjusted to position the air inlet tubes 59 and their respective valve outlets 60 at the desired positions along the outside surfaces of the liner 50 to provide maximum inflation accessibility and/or comfort to the wearer. The lower strap 30a is positioned over the tongue portion 35b or threaded through the spaced slots 37 of the lower portion 35b of the tongue 35 and through the D ring 25a, to position the lower tongue portion 35b over the foot portion of the wearer. The strap 30b is pulled across the forward surface of the tongue 35 and threaded through the D ring 25b. As the strap 30b is pulled through the D ring 25b pressure is applied through the tongue 35 and the forward portion of the liner 40, to urge and secure the wearer's foot and ankle toward the back heel portion of the brace. The strap 30b is folded back upon itself to secure its Velcro fasteners, and thus the strap 30b, in the desired fastened position. The three upper straps 42, 43, and 44 are extended to overlie the forward surface of the upper portion 35a of the tongue 35 and are threaded through their respective D rings 45 to apply rearward and compressive pressure to the wearer's ankle and leg through the tongue 35 and the elongate upright stirrup supports. The straps 42, 43 and 44 are tightened to the desired pressure and fastened in the same manner as strap 30b by folding the straps respectively over onto themselves to secure their hook and loop fastening structures. The strap 30a is tightened in similar manner by folding upon itself to apply the desired pressure to the forward foot portion of the wearer. The individual straps can be selectively loosened and tightened to provide the desired pressures to the wearer's foot, ankle and leg portions being retained by the brace. Finally, the air bladders 58 and be pressurized to the desired pressure by pumping air into them with a pump such as the bulb pump 62, through the valve assemblies 60, as previously described.

It will be understood that the forgoing description and accompanying drawings relate to a preferred embodiment of the invention. Various modifications may be employed without departing from the spirit and scope of the invention. Further, while various preferred materials have been described with respect to construction of the walker brace, it will be understood that other comparable materials that perform the desired functions of the brace can be used within the scope of the invention. Further, while various fastening mechanisms have been illustrated to secure the various parts of the walker brace to one another, it will be understood that other such fastening mechanisms which perform the desired functions could be used. It will also be appreciated that the unique two part construction of the side upright portions 15 and 16 of the stirrup 12 enables the length of the stirrup sides to be shortened if desired, by detaching the upper portions 15a and 15b respectively from their associated lower portions 16a and 16b. If such a low-top configuration were to be employed, appropriate “shorter” tongue and back panel members could be provided to accommodate the shorter height of the brace.

The above specification and examples provide a complete description of the manufacture and use of the composition of the invention. Since many embodiments of the invention can be made without departing from the spirit and scope of the invention, the invention resides in the claims hereinafter appended.

1. A walking brace for providing therapeutic pressure to the ankle and lower leg of a person, comprising:
a. an exterior stirrup frame construction, comprising:
i. a full length sole portion; and
ii. a pair of rigid or semirigid generally parallel elongate strut members extending substantially vertically upward from said sole portion on either side thereof so as to extend along an ankle and leg of a person wearing said brace;
b. an interior resilient liner pad configured to cushion the ankle and leg of a person and to cooperatively position the leg within said stirrup frame;
c. at least one detachable variable pressure fluid bladder;
d. at least one bladder support pocket attached to said liner pad configured to support said at least one fluid bladder, said support pocket having an access suitable for enabling ingress and egress of said bladder therethrough;
e. a fluid inlet tube operatively connected to and extendable from said fluid bladder to a position remote therefrom and accessible externally of said liner and said stirrup frame; and
f. a fastener operatively connectable to said inlet tube to selectively secure said inlet tube at a plurality of locations relative to said liner and said stirrup frame.

2. The walking brace of claim 1, wherein said fastener is configured to attach to said liner.

3. The walking brace of claim 1, wherein said fastener comprises a sleeve member removably attachable to said fluid inlet tube.

4. The walking brace of claim 1, wherein said fastener comprises at least in part, a hook or loop fastening member.

5. A walking brace as recited in claim 1, including a plurality of brace fasteners connected to said stirrup frame and configured to apply selective pressure through said frame to said ankle and leg.

6. The walking brace as recited in claim 5, wherein said brace fasteners comprise a plurality of straps.

7. A walking brace as recited in claim 1, further including a rigid or semirigid tongue member configured to cooperatively overlie said liner adjacent a front portion of said stirrup frame.

8. The walking brace as recited in claim 1, including a detachable back support member generally connectable between said elongate strut members near a back portion of said stirrup frame.

9. The walker brace as recited in claim 1, wherein said sole portion comprises a padded upper platform configured to support the bottom surface of a person's foot.

10. The walking brace as recited in claim 1, wherein said sole portion further comprises a curved floor engaging lower surface to provide a rocker walking surface for said brace.

11. The walking brace as recited in claim 8, wherein including a plurality of brace fasteners connected to said stirrup frame and configured to apply selective pressure through said frame to said ankle and leg, said brace fasteners connecting said upright strut members to said back support member.

12. The walking brace of claim 1, wherein said elongated strut members are each configured in bifurcated detachable manner so as to enable shortening or lengthening of said strut member.

13. An inflatable fluid bladder support member for selectively applying pressure to an ankle and leg of a patient and of a type suitable for placement between an outer shell of an orthopedic brace member and the ankle or leg of a patient, comprising:
a. a fluid inflatable bladder; having an internal cavity;
b. an inlet tube having a first end operatively connected to said bladder in fluid communication with said bladder internal cavity, and extending to an opposite second end suitable for selective attachment to a pump; and
c. a fastener member secured to said tube between said first and second ends thereof and having an outer surface suitable for detachable connection to a second attachment surface; said outer surface being attachable at an infinite number of positions along said second attachment surface.

14. The bladder support member of claim 13, wherein said fastener member comprises a tubular sleeve member slidably attached to said tube and having an outer surface comprising a hook or loop fastener material.

15. The bladder support member of claim 14 wherein said tubular sleeve member extends along substantially the entire length of said tube between said first and said second ends thereof.
